(19) United States
(12) Patent Application Publication
(10) Pub. No.: US 2005/0159706 A1
(43) Pub. Date: Jul. 21, 2005

(54) Medical syringe with safety shield system

(75) Inventors: Bradley Wilkinson, North Haledon, NJ (US); Frank Francavilla, Branchville, NJ (US)

Correspondence Address: COHEN, PONTANI, LIEBERMAN & PAVANE, 551 FIFTH AVENUE, SUITE 1210, NEW YORK, NY, 10176, US

(73) Assignee: Becton, Dickinson and Company

(21) Appl. No.: 10/760,883

(22) Filed: Jan. 20, 2004

(51) Int. Cl. A61M 5/00

(52) U.S. Cl. 604/110; 128/919

(57) ABSTRACT

A medical device for delivering a medicament to a patient includes a syringe assembly having a barrel defining a reservoir containing the medicament, a needle cannula coupled to a forward end of the barrel, and a plunger having a stopper positioned in the barrel and movable into the barrel to cause the medicament to be expelled. The medical device also includes a cap arranged on, and slidable over, the needle cannula from a first position in which the forward tip of the needle cannula is exposed, to a second position in which the forward tip of the needle cannula is covered by the cap. An actuation mechanism connected to the cap includes an urging member coupled to the barrel and the cap for urging the cap toward the second position. A trigger element releasably secures the urging member in a charged state and releasably secures the cap in the first position. The trigger element is actuatable to release the cap by either manual actuation or by interaction with the thumb pad.

### BACKGROUND OF THE INVENTION

1. Field of the Invention

The present invention relates to a prefilled medical device for delivering a dose of medicament by injection and having an integral shield system for preventing accidental needle sticks after use. More particularly, the present invention is directed to a syringe assembly including a safety shield system.

2. Description of the Related Art

Syringes used for the delivery of medicaments to patients are well known. Oftentimes syringes are prefilled with a dosage of a medicament or other substance by a pharmaceutical manufacturer and then distributed to end users such as health care professionals or patients for administration of the prefilled medicament. Such syringes typically include a cylindrical hollow barrel which may be formed of a glass or plastic material and which includes the medicament. One end of the barrel is fitted with a fixed or removable hollow needle, and the other end of the barrel receives a plunger having a stopper which is slidable with respect to the barrel for delivery of the medicament to the hollow needle, i.e., to urge the medicament toward and out of the needle. A syringe assembly, which typically includes the above-described components, is usually stored with a removable needle cover which protects the needle from damage during storage and handling. Prior to use, the needle cover is removed to expose the needle.

To prevent a syringe user and, in particular, a health care professional from inadvertent sticks by the needle after use of the syringe on a patient, the syringe assembly may incorporate a safety shield which forms a guard to cover the needle after use. Certain attributes to be considered in such syringe assemblies are that the shield should be intuitive and easy to use, should preferably provide consistent and reliable shield deployment, and should be operable with one hand. Other attributes are that such syringe assemblies require no change in current medicament delivery techniques, allow for dose adjustment, are preferably autoclavable, and allow for the inspection of contents before and after activation of the shield. Moreover, the use of the shield must not detrimentally affect processing and filling of the syringe at the pharmaceutical company, the assembly (i.e., syringe assembly and safety shield) must be easy to manufacture, must prevent accidental activation, and must limit the possibility of incurring cosmetic or structural damages.

### SUMMARY OF THE INVENTION

The present invention relates to a syringe assembly incorporating a safety shield for covering the needle of the syringe assembly after administration of a dosage of medicament. The safety shield is automatically activated upon full delivery of the medicament dosage in the syringe.

According to an embodiment of the present invention, a medical device for delivering a medicament to a patient includes a syringe assembly comprising a barrel having a forward end and a rear end and defining a reservoir within which the medicament may be contained, a needle cannula having a forward tip which is coupled to the forward end of the barrel and in fluid communication with the reservoir, and a plunger having a first end with a stopper positioned in the reservoir and a second end having a thumb pad or thumb press area for receiving medicament delivery pressure for causing the plunger to move within the reservoir to cause the medicament to be expelled from the reservoir and through the needle cannula.

A cap is arranged on said needle cannula such that it is slidable along the needle cannula from a first position in which the forward tip of the needle cannula is exposed, to a second position in which the forward tip of the needle cannula is covered by the cap. The cap is connected to an actuation mechanism which includes an urging member coupled to the barrel and the cap, and a trigger element releasably securing the urging member in a charged state and releasably securing the cap in the first position. The trigger element is actuatable to release the urging member and cap by one of manual actuation or interaction with the thumb pad.

The thumb pad may be configured to interact with the trigger element upon movement of the plunger rod to a fully inserted position, in which the stopper is proximate the forward end of the syringe barrel, to release the urging element and the cap for allowing the cap to move to the second position by the urging member. The trigger element may include an actuator rod connected to the cap and extending longitudinally along a length of the barrel. The barrel may further define a secondary barrel through which the actuator rod is inserted. The actuator rod further comprises a first radial projection which interacts with a constriction of the cross-section of the secondary barrel to releasably secure the cap in the first position. In this embodiment, the thumb pad interacts with the actuator rod to move the first radial projection through the constriction upon movement of the plunger rod to the fully inserted position. This releases the urging element and the cap and allows the cap to be moved to the second position by the urging member.

The urging member may comprise a spring having one end supported on a support defined in the secondary barrel and another end supported on a second radial projection on the actuator rod. The support defined in the secondary barrel may be the constriction described above. The secondary barrel may further define a blocking element for blocking forward movement of the actuator rod when the cap is in the second position. The blocking device may interact with the second radial projection on which the spring is supported.

The cap defines a through-hole through which the needle cannula extends. A slot extending from the through-hole is arranged on a rear-facing side of the cap. The cap is biased against the needle cannula such that the forward tip of the needle cannula enters the slot when the cap is in the second position. Alternatively, the cap may include a resiliently mounted latching element which rests against the needle cannula in the first position and blocks the through-hole in the cap in the second position of the cap, thereby preventing the cap from moving back toward the first position.

In yet another embodiment, the urging element may comprise a biasing arm connected between the spring barrel and the cap.

The trigger element may include a blocking element device releasably engaging the cap for holding the cap in the first position against the urgency of the urging member. In this embodiment, the trigger element may further include an actuator rod connected to the blocking element. The thumb pad interacts with the actuator rod to move the actuator rod when the plunger is in the fully inserted position such that the blocking element is released and the cap is allowed to move to the second position by the urging member. Alternatively, the blocking device may be manually releasable for allowing the cap to be moved to the second position by the urging member.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings, wherein like reference characters denote similar elements throughout the several views:

FIG. 1 is an exploded perspective view of an embodiment of a medical device according to the present invention;

FIG. 2 is a longitudinal sectional view of the medical device of FIG. 1;

FIG. 2a is a partial sectional view of another embodiment of the present invention;

FIG. 2b is a partial sectional view of yet another embodiment of the present invention;

FIG. 3 is a side view of the medical device of FIG. 1 during delivery of a medicament to a patient;

FIG. 4 is a side view of the medical device of FIG. 1 after full delivery of the medicament and prior to withdrawal from the patient;

FIG. 5 is an enlarged view of a front end of the medical device after use;

FIG. 6 is a perspective view of a medical device according to another embodiment of the present invention;

FIG. 6a is a partial sectional view of an actuator rod of the medical device of FIG. 6;

FIG. 7 is a longitudinal sectional view of a front end of the medical device of FIG. 6 prior to use;

FIG. 8 is a partial sectional side view of the device according to FIG. 7;

FIG. 9 is a partial sectional side view of the device according to FIG. 7 after use thereof; and

FIG. 10 is a side view of still another embodiment of the present invention.

### DETAILED DESCRIPTION OF THE PRESENTLY PREFERRED EMBODIMENTS

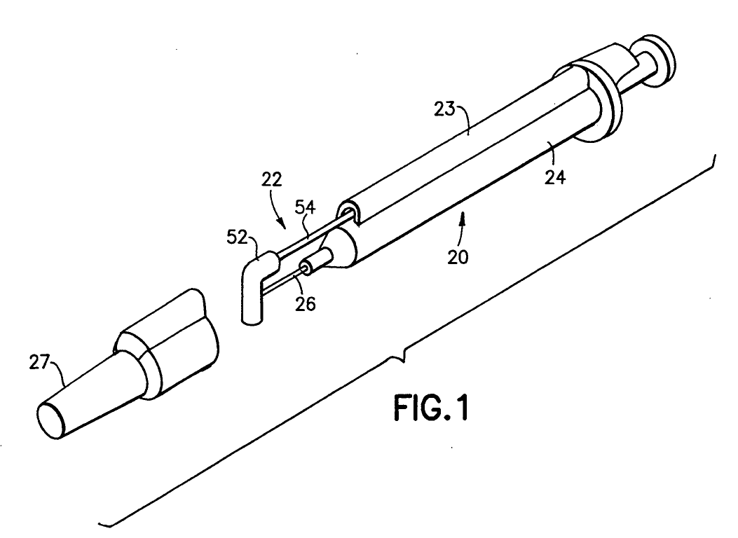

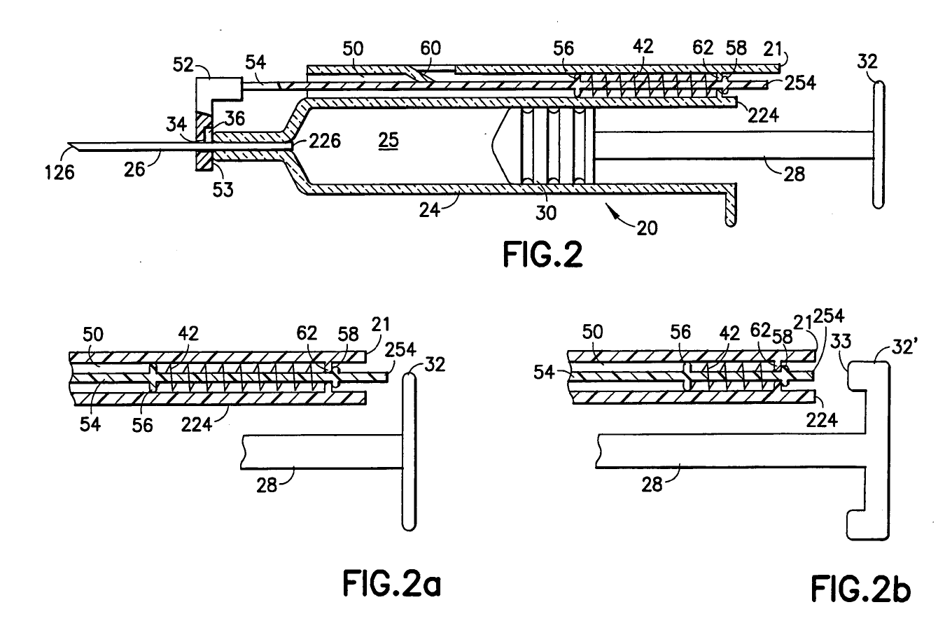

FIGS. 1 and 2 show a medical device 10 for delivery of a medicament into a patient constructed in accordance with an embodiment of the present invention. As used herein, the term “medicament” is intended to refer to any drug substance, vaccine, or other liquid substance that is injected into a patient. The medical device 10 includes a syringe assembly 20 which can be prefilled with the medicament to be delivered, and a shield cap assembly 22.

The syringe assembly 20 includes a cylindrical syringe barrel 24 defining a reservoir 25 within which the medicament is held prior to use of the medical device 10. The syringe assembly 20 also includes a needle cannula 26 having a forward tip 126 and a rearward end 226 in fluid communication with the reservoir 25. The needle cannula 26 may be permanently connected to a front end of the syringe barrel 24 using an adhesive, glue, interference fit or other known or hereafter developed material or technique, or it may be detachable from the syringe barrel 24 such as, for example, using a luer-type connection. A plunger rod 28 has a first end inserted in the syringe barrel 24 with a stopper or piston 30 arranged on the first end that is movable with the plunger rod 28 within the syringe barrel 24. A second end of the plunger rod 28 includes a thumb pad or thumb press area 32 used for receiving pressure from the user's thumb for moving the piston 30 into and within the syringe barrel 24. As further shown in FIG. 1, a removable needle shield 27 may be disposed over the needle cannula 26 on the front end of the syringe barrel 24 to protect the needle from damage during handling of the syringe assembly, and to protect users from being stuck by the needle prior to its intended use. The needle shield 27 is not a conventional shape and includes a protuberance on one side thereof so as to accommodate the shield cap assembly 22. The needle shield 27 preferably includes a pliable part and a rigid part.

A secondary barrel 23 is mounted on the syringe barrel 24 defining a longitudinal space 50 therethrough having an end 21. The secondary barrel 23 may be made of plastic or glass and attached to the syringe barrel 24 using an adhesive or glue or any known or hereafter developed material or technique. The syringe barrel 24 may also be made of plastic or glass. The syringe barrel 24 and secondary barrel 23 may be made of two separate materials. Alternatively, the secondary barrel 23 may be formed integrally with the syringe barrel 24.

An actuator rod 54 having an actuator end 254 is received in the longitudinal space 50 defined in the secondary barrel 23. A front end of the actuator rod 54 is connected to a sliding cap 52 which is movable on the needle cannula 26 between a first position as shown in FIG. 2, in which the forward tip 126 of the needle cannula 26 is exposed, and a second position in which the forward tip 126 is covered by the sliding cap 52 (described below). The actuator rod 54 extends through said secondary barrel 23 and past a rear end 224. An urging member 42, such as, for example, a coil spring or a biasing arm, is positioned within the longitudinal space 50 in the secondary barrel 23 between a spring support 62 on the secondary barrel 23 and a radial flange 56 arranged on the actuator rod 54. The spring 42 is fully charged or compressed in the state shown in FIG. 2 such that the actuator rod 54 and sliding cap 52 connected thereto are biased forward toward the second position of the sliding cap 52. A radial projection or bulge 58, which may comprise an annular bump on the actuator rod 54, interacts with the spring support 62 and forms a constriction in the longitudinal space 50 to prevent the actuator rod 54 from prematurely moving toward the second position.

As shown in FIG. 2, rear end 254 of the actuator rod 54 is opposite the thumb pad 32 but does not extend toward the thumb pad passed end 21 of secondary barrel 23. Most preferably, end 21 extends a sufficient distance beyond rear end 254 (in a direction toward thumb pad 32) so as to prevent a user of the device 10 from inadvertently pushing rear end 254 to prematurely deploy the sliding cap 52. For this purpose, thumb pad 32 is dimensioned to engage rear end 254 without contacting, and consequently being obstructed by, secondary barrel end 21.

As an alternative to the embodiment depicted in FIG. 2, rear end 254 may extend beyond secondary barrel end 21 as shown in FIG. 2a so that thumb pad 32 can engage rear end 254 in the intended manner as explained below. In this embodiment, however, the inclusion of a safety feature is desired to prevent accidental activation of the actuator rod 54 and, hence, unintended deployment of the sliding cap 52.

As a further alternative shown in FIG. 2b, end 21 of secondary barrel 23 may be coterminous with, or extend beyond, rear end 254 and a specifically configured thumb pad 32′ may be incorporated to actuate the rear end 254 of the actuator rod 54. For example, thumb pad 32′ will include a depending section 33 dimensioned for insertion within the open end of secondary barrel 23, i.e. at the end of space 50, to engage rear end 254 and cause deployment of the sliding cap 52.

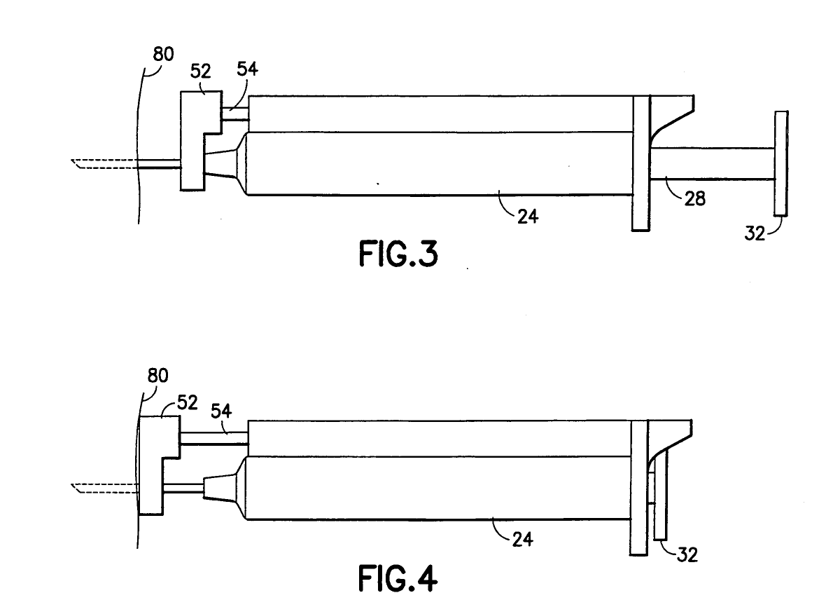

During use of the syringe the needle cannula 26 is inserted into a patient and the thumb pad 32 of the plunger rod 28 is depressed to deliver the medicament to the patient (see FIG. 3). Once the plunger rod 28 is pushed to a fully inserted position in which the stopper 30 is proximate the front end of the barrel 24, the thumb pad 32 pushes the rear end 254 of the actuator rod 54 (see FIG. 2) such that the bulge 58 of the actuator rod 54 is pushed through the spring support 62. Although the bulge 58 has a larger diameter than the remainder of the actuator rod 54, it is small enough to move through the spring 42 with minimal friction and the urgency of the spring 42 pushes the actuator rod 54 and sliding cap 52 toward the second position. As shown in FIG. 4, when the needle cannula 26 is in the patient, the patient's skin prevents the sliding cap 52 from moving all the way to the second position.

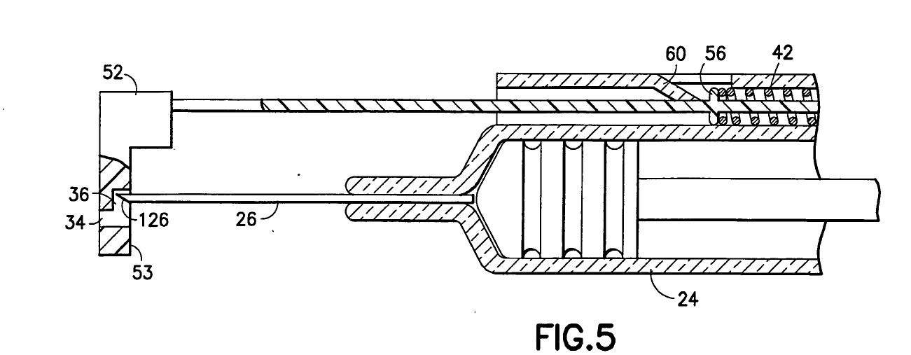

When the needle cannula 26 is withdrawn from the patient, the patient's skin no longer obstructs forward movement of the sliding cap 52, and the sliding cap 52 then moves to the second position as shown in FIG. 5. The sliding cap 52 is prevented from moving past the forward tip 126 of the needle cannula 26 by a blocking device 60 arranged in the secondary barrel 23 which interacts with the radial flange 56 to prevent further forward movement when the sliding cap 52 reaches the second position. As shown in FIGS. 2 and 5, the sliding cap 52 has a through hole 34 through which the needle cannula 26 extends in the first position. A slot 36 is also arranged on a rear side 53 of the sliding cap 52 facing the syringe barrel 24. Once the forward tip 126 of the needle cannula 26 enters the axial extent of the slot 36, the sliding cap 52 moves so that the needle cannula 26 is not aligned with the through-hole 34. To accomplish this movement, the actuator rod 54 is biased downward or toward the needle cannula. Of course, the slot could also be configured on the other side of the through-hole 34. In that case, the actuator rod 54 would be biased away from the needle cannula 26. The misalignment of the needle cannula 26 with the through-hole 34 prevents the needle cannula 26 from extending back out of the through-hole 34 after use. Furthermore, the sliding cap 52 must be made of sufficiently strong material to prevent the forward tip 126 of the cannula 26 from piercing through the sliding cap 52 when the forward tip is in the slot 36. A further blocking device (not shown) may be arranged in the secondary barrel 23 to act on radial flange 56 when the sliding cap 52 reaches the second position to prevent the actuator rod 54 and sliding cap 52 from moving rearward. This additional blocking device may be similar to blocking device 60 acting in a reverse direction and may be used in addition to or as an alternative to the slot 36.

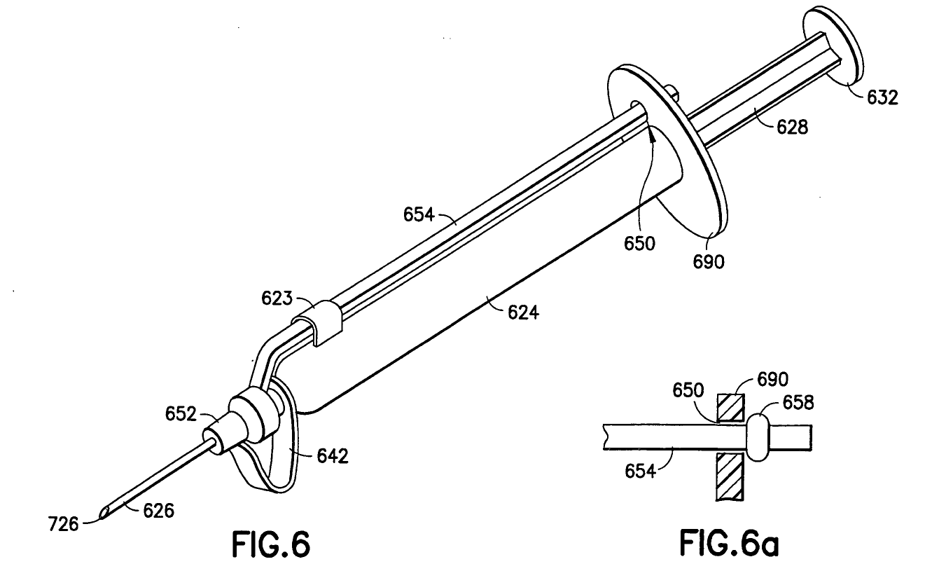

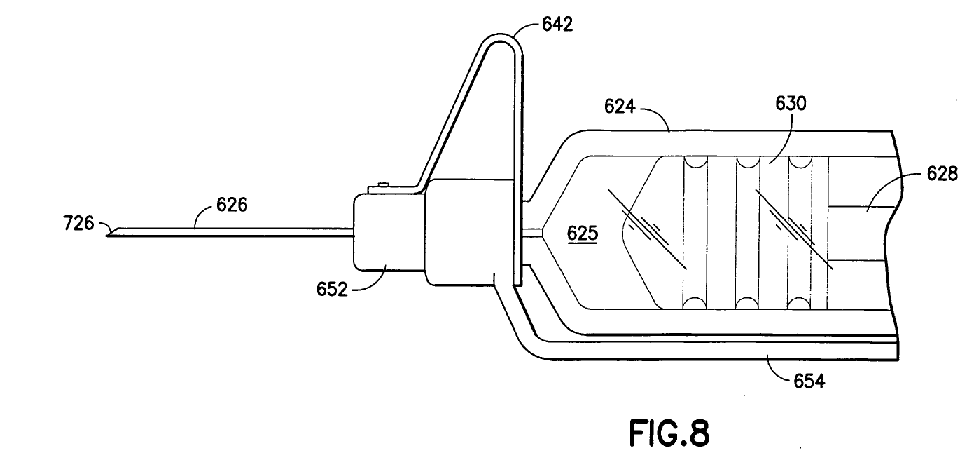

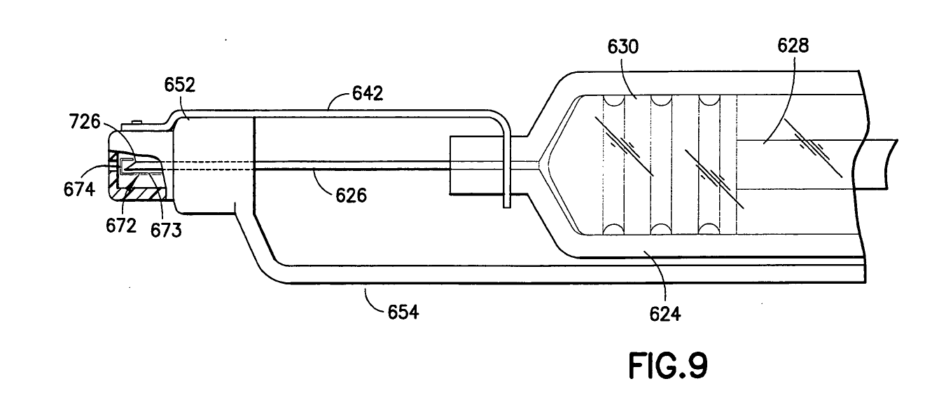

A further embodiment of the present invention shown in FIGS. 6-9 also includes a syringe barrel 624 having a needle cannula 626 and a plunger 628 with a thumb pad 632 as described above. An actuator rod 654 is connected to a sliding cap 652 which is slidable on the needle cannula 626 from a first position shown in FIG. 6 in which the forward tip 726 of the needle cannula is exposed and a second position (see FIG. 9) in which the cap 652 covers the forward tip of the needle cannula 626. The sliding cap 652 has a through hole 634 through which the needle cannula 626 extends. In this embodiment, an urging member 642 comprising a biasing arm is connected between the barrel 624 and the sliding cap 652. The biasing arm 642 is resiliently bent such that it exerts a force on the sliding cap 652 directed toward the second position. As best shown in FIG. 6a, the actuator rod 654 extends through a hole 650 in a flange 690 on the exterior of the syringe barrel 624. The actuator rod 654 has a bulge 658 that interacts with the flange 690 such that the sliding cap 652 is prevented from moving toward the second position. When the medicament is fully delivered, the thumb pad 632 pushes the bulge 658 through the flange 690, thereby allowing the biasing arm 642 to move the sliding cap 652 to the second position. The actuator rod 654 is inserted through a holder 623 connected to the syringe barrel 624 for guiding the actuator rod 654 as the actuator rod 654 and the sliding cap 652 move toward the second position.

The biasing arm 642 has one end coupled to the syringe barrel 624 and another end coupled to the sliding cap 652. When the actuator rod 654 is actuated by the thumb pad 632, the biasing arm moves the sliding cap 652 along the needle cannula 626 until the sliding cap 652 covers the forward tip 726 of the needle cannula 626 as shown in FIG. 9. The length of the biasing arm 642 prevents the sliding cap from sliding off of the needle cannula 626. Furthermore, the sliding cap also includes a latching mechanism 672 for preventing the sliding cap from moving back onto the needle cannula 626 and uncovering the forward tip of the needle cannula 626.

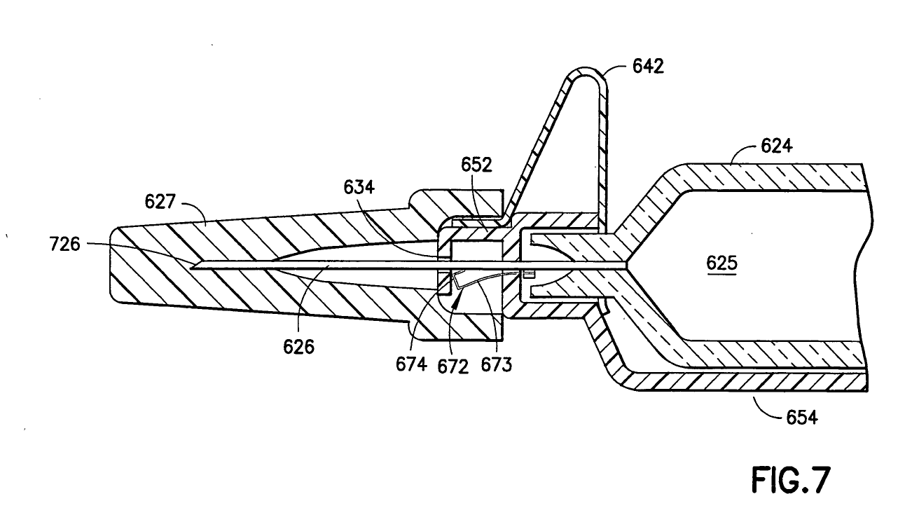

As shown in FIGS. 7 and 9, the latching mechanism 672 is arranged within the sliding cap 652 and comprises a lever 673 biased against the needle cannula 626 when the sliding cap is in the first position (FIG. 7). When the sliding cap 652 is moved to the second position, the lever 673 moves and a blocking element 674 is positioned over the end of the needle cannula 626 preventing the sliding cap 652 from moving back toward the first position. Alternatively, the sliding cap 652 could be biased by the biasing arm such that the needle cannula 626 is moved out of alignment with the hole 634 in the second position as in the embodiment of FIGS. 1-5.

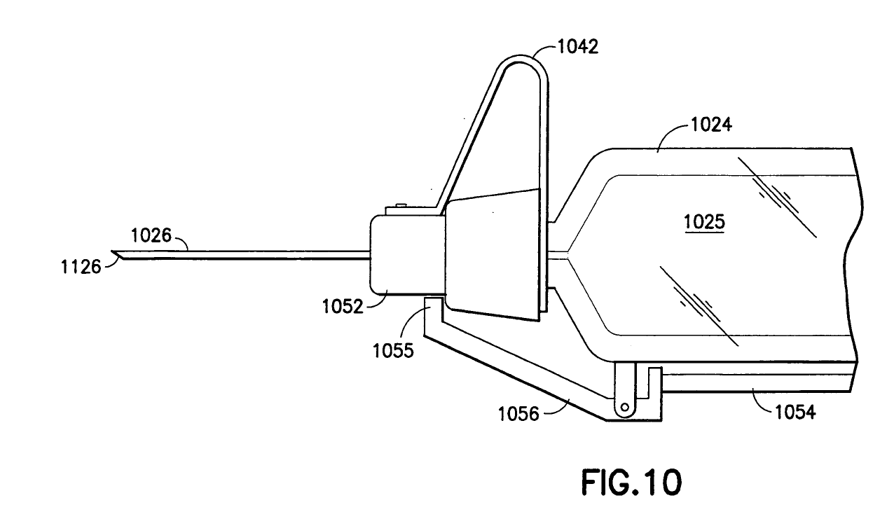

Instead of having the actuator rod 54, 654 connected to the sliding cap 52, 652, FIG. 10 shows an embodiment in which an actuator rod 1054 is connected to a blocking device or retention member 1055 which selectively holds the sliding cap 1052 in the first position against the urgency of the biasing member 1042. In this embodiment, the blocking device 1055 is connected to a lever 1056 that is pivotally mounted on the syringe barrel 1054. When the thumb pad (not shown in FIG. 10) interacts with the actuator rod 1054, the lever 1056 pivots and the blocking device 1055 releases the sliding cap 1052 and allows the sliding cap 1052 to be moved to the second position under the urgency of the biasing member 1042, in which the sliding cap 1052 covers the forward tip 1126 of the needle cannula 1026.

Instead of automatically actuating the actuator rod 1054 by the thumb pad 32, 632 upon delivery of the full dose of medicament, the actuator rod 1054 may be manually actuatable. In this alternative embodiment, the actuator rod 1054 is manually actuated by a user after the medicament has been fully delivered. The manual actuation may be performed while the needle cannula is in a patient or after it has been withdrawn from the patient.

A description of an exemplary usage of the medical device 10 of the present invention will now be provided. It should be understood by a person of ordinary skill in the art that the following description is provided as an illustrative and non-limiting example. The health care professional receives the inventive medical device 10 prefilled with a desired single dosage of a medicament. Immediately prior to use, the needle shield 27 is removed and the needle cannula 26 and forward tip 126 are exposed. The health care professional pierces the patient's skin with the forward tip 126 of the needle cannula 26 and depresses the thumb pad 32 to cause the plunger rod 28 and piston 30 to move within the reservoir 25. As the plunger rod 28 and piston are caused to move into the reservoir 25, the medicament is caused to be expelled from the reservoir, through the needle cannula 26, and into the patient. When the medicament is completely expelled from the reservoir (i.e., the dose has been completely administered), the thumb pad 32 interacts with the actuator rod 54, thereby releasing the sliding cap 52 and enabling the sliding cap 52 to move from the first position to the second position under the force of the spring or urging member 42. When in the second position, the forward tip 126 of the needle cannula 24 will be completely contained within the sliding cap 52, thus preventing undesired and inadvertent exposure of the health care professional to the contaminated forward tip 126. The used medical device 10 may then be disposed of in a suitable sharps disposal container.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements which perform substantially the same function in substantially the same way to achieve the same results are within the scope of the invention. Specifically, the biasing arm 642 may be used instead of the spring 42 in the embodiment of FIGS. 1-5 and the spring 42 may be used in place of the biasing arm 642 in FIGS. 6-9. Furthermore, the lever 672 may be incorporated in the cap 52 of FIGS. 1-5 and the through-hole 34 and slot 36 configuration may be used in the sliding caps 652 and 1052 of FIGS. 6-10. Moreover, it should be recognized that structures and/or elements shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A medical device for delivering a medicament to a patient, comprising:
a syringe assembly comprising:
a barrel having a forward end and a rear end and defining a reservoir within which the medicament may be contained;
a needle cannula having a forward tip and being coupled to said forward end of said barrel and in fluid communication with said reservoir; and
a plunger having a first end with a stopper positioned in said reservoir and a second end having a thumb pad for receiving medicament delivery pressure for causing said plunger to move within said reservoir to cause the medicament to be expelled from said reservoir;
a cap arranged on said needle cannula and slidable along said needle cannula from a first position in which said forward tip of said needle cannula is exposed, to a second position in which said forward tip of said needle cannula is covered by said cap; and
an actuation mechanism connected to said cap, said actuation mechanism having an urging member coupled to said barrel and said cap, and a trigger element releasably securing said urging member in a charged state and releasably securing said cap in said first position, said trigger element being actuatable to release said urging member and said cap by one of manual actuation or interaction with said thumb pad.

2. The medical device of claim 1, wherein said thumb pad is configured to interact with said trigger element upon movement of said stopper to a position proximate said syringe barrel forward end to release said urging element and said cap for allowing said cap to move to said second position by said urging member.

3. The medical device of claim 2, wherein said trigger element comprises an actuator rod connected to said cap and extending longitudinally along a length of said barrel.

4. The medical device of claim 3, wherein said barrel further defines a secondary barrel having a substantially even cross-section, said actuator rod being received in said secondary barrel.

5. The medical device of claim 4, wherein said actuator rod further comprises a first radial projection which interacts with a constriction of said cross-section in said secondary barrel to releasably secure the cap in said first position.

6. The medical device of claim 5, wherein said thumb pad interacts with said actuator rod to move said first radial projection through said constriction upon movement of said stopper to a position proximate said forward end of said syringe barrel, thereby releasing said urging element and said cap and allowing said cap to be moved to said second position by said urging member.

7. The medical device of claim 5, wherein said urging member comprises a spring having one end supported on a support defined in said secondary barrel and another end supported on a radial projection on said actuator rod.

8. The medical device of claim 7, wherein said support comprises said constriction.

9. The medical device of claim 7, wherein said secondary barrel further defines a first blocking element for blocking forward movement of said actuator rod when said cap is in said second position.

10. The medical device of claim 9, wherein said secondary barrel further defines a second blocking element for blocking rearward movement of said actuator rod when said cap is in said second position.

11. The medical device of claim 9, wherein said blocking device interacts with said radial projection.

12. The medical device of claim 1, wherein said cap defines a through-hole and a slot extending from said through-hole on a rear-facing side of said cap, wherein said cap is biased against said needle cannula such that said forward tip of said needle cannula enters said slot when the cap is in said second position.

13. The medical device of claim 1, wherein said cap further comprises a resiliently mounted latching element which rests against said needle cannula in said first position and blocks the through-hole in said cap in the second position of said cap, thereby preventing said sliding cap from moving back toward said first position.

14. The medical device of claim 1, wherein said urging element comprises a biasing arm connected between said barrel and said sliding cap.

15. The medical device of claim 14, wherein said cap defines a through-hole and a slot extending from said through-hole on a rear-facing side of said cap, wherein said cap is biased against said needle cannula such that said forward tip of said needle cannula enters said slot when the cap is in said second position.

16. The medical device of claim 14, wherein said cap further comprises a resiliently mounted latching element which rests against said needle cannula in said first position and blocks the through-hole in said cap in the second position of said cap, thereby preventing said sliding cap from moving back toward said first position.

17. The medical device of claim 14, wherein said trigger element includes a blocking element releasably engagable with said cap device for holding said cap in the first position against the urgency of the urging member.

18. The medical device of claim 17, wherein said trigger element further includes an actuator rod connected to said blocking element, wherein said thumb pad interacts with said actuator rod to move said actuator rod upon movement of said stopper to a position proximate said forward end of said syringe barrel, thereby releasing said blocking element and allowing said cap to be moved to said second position by said urging member.

19. The medical device of claim 17, wherein said blocking device is manually releasable for allowing said cap to be moved to said second position by said urging member.

20. The medical device of claim 1, wherein said urging member comprises a spring acting between said barrel and said cap.

21. The medical device of claim 1, wherein said barrel is plastic.

22. The medical device of claim 1, wherein said barrel is glass.

23. A combination comprising a medical syringe with a safety shield, said medical syringe comprising a barrel having a forward end and a rear end and defining a reservoir within which the medicament may be contained, a needle cannula having a forward tip and being coupled to said forward end of said barrel and in fluid communication with said reservoir, and a plunger having a first end with a stopper positioned in said reservoir and a second end having a thumb pad for receiving medicament delivery pressure for causing said plunger to move within said reservoir to cause the medicament to be expelled from said reservoir; and
said safety shield comprising a cap arranged on said needle cannula and slidable along said needle cannula from a first position in which said forward tip of said needle cannula is exposed, to a second position in which said forward tip of said needle cannula is covered by said cap, and
an actuation mechanism connected to said cap, said actuation mechanism having an urging member coupled to said barrel and said cap, and a trigger element releasably securing said urging member in a charged state and releasably securing said cap in said first position, said trigger element being actuatable to release said cap by one of manual actuation or interaction with said thumb pad.

24. The combination of claim 23, wherein said thumb pad is configured to interact with said trigger element upon movement of said stopper to a position proximate said syringe barrel forward end to release said urging element and said cap and allowing said cap to move to said second position by said urging member.

25. The combination of claim 24, wherein said trigger element comprises an actuator rod connected to said cap and extending longitudinally along a length of said barrel.

26. The medical device of claim 25, wherein said safety shield assembly further comprises a secondary barrel connected to said syringe barrel, said secondary barrel having a substantially constant cross-section, said actuator rod being received in said secondary barrel.

27. The medical device of claim 26, wherein said actuator rod further comprises a first radial projection which interacts with a constriction of said cross-section in said secondary barrel to releasably secure the cap in said first position.

28. The combination of claim 27, wherein said urging member comprises a spring having one end supported on a support defined in said secondary barrel and another end supported on a radial projection on said actuator rod.

29. The combination of claim 27, wherein said secondary barrel further defines a blocking element realeasably engagable with said cap for blocking forward movement of said actuator rod when said cap is in said second position.

30. The combination of claim 23, wherein said cap defines a through-hole and a slot extending from said through-hole on a rear-facing side of said cap, wherein said cap is biased against said needle cannula such that said forward tip of said needle cannula enters said slot when the cap is in said second position.

31. The combination of claim 23, wherein said cap further comprises a resiliently mounted latching element which rests against said needle cannula in said first position and blocks the through-hole in said cap in the second position of said cap, thereby preventing said sliding cap from moving back toward said first position.

32. The combination of claim 23, wherein said urging element comprises a biasing arm connected between said spring barrel and said sliding cap.

33. The combination of claim 23, wherein said urging member comprises a spring acting between said barrel and said cap.

34. The combination of claim 23, wherein said barrel is plastic.

35. The combination of claim 23, wherein said barrel is glass.

36. A medical device for delivering a medicament to a patient, comprising:
a syringe assembly comprising:
a barrel having a forward end and a rear end and defining a reservoir within which the medicament may be contained;
a needle cannula having a forward tip and being coupled to said forward end of said barrel and in fluid communication with said reservoir; and
a plunger having a first end with a stopper positioned in said reservoir and a second end having a thumb pad for receiving medicament delivery pressure for causing said plunger to move within said reservoir to cause the medicament to be expelled from said reservoir;
a cap arranged on said needle cannula and slidable along said needle cannula from a first position in which said forward tip of said needle cannula is exposed, to a second position in which said forward tip of said needle cannula is covered by said cap; and
an actuation mechanism connected to said cap, said actuation mechanism having means for urging said cap toward said second position, means for releasably securing said urging means in a charged state and means for releasably securing said cap in said first position, said means for releasably securing being actuatable to release said cap by one of manual actuation or interaction with said thumb pad.
